(19) United States
(12) Patent Application Publication
(10) Pub. No.: US 2014/0336522 A1
(43) Pub. Date: Nov. 13, 2014

(54) INFORMATION PROCESSING APPARATUS AND REPRESENTATIVE-WAVEFORM GENERATING METHOD

(71) Applicant: FUJITSU LIMITED, Kawasaki-shi (JP)

(72) Inventors: Yasuyuki NAKATA, Zama (JP); Akihiro INOMATA, Atsugi (JP)

(21) Appl. No.: 14/444,683

(22) Filed: Jul. 28, 2014

Related U.S. Application Data
(63) Continuation of application No. PCT/JP2012/052302, filed on Feb. 1, 2012.

(51) Int. Cl. A61B 5/0456

(52) U.S. Cl. CPC A61B 5/0456
USPC 600/521

(57) ABSTRACT

A handheld terminal includes an electrocardiographic-signal dividing module that divides a biosignal into waveforms of a fixed interval, an R-R interval calculating module that calculates a plurality of R-R intervals indicative of an interval between adjacent R-waves and calculates an average value of the calculated R-R intervals for each of the waveforms of the fixed interval divided, and a candidate-waveform selecting module that selects a plurality of waveforms of a fixed interval corresponding to average values indicating near a maximum value of frequency of average values using the average value of the R-R intervals calculated for each of the waveforms of the fixed interval calculated, and thus highly accurate representative waveform data can be generated.

### CROSS-REFERENCE TO RELATED APPLICATION(S)

This application is a continuation of International Application No. PCT/JP2012/052302, filed on Feb. 1, 2012, the entire contents of which are incorporated herein by reference.

### FIELD

The embodiments discussed herein are directed to information processing apparatuses, for example.

### BACKGROUND

In recent years, mechanisms have been developed to monitor a biosignal at all times with a simple sensor that can be attached to a body. Such mechanisms, however, are susceptible to noise for reasons that the number of electrodes attached to the body is small and a signal processing circuit is simplified, for example. Thus, desired is a method that reduces the influence of noise to acquire a biosignal with high accuracy.

For example, disclosed as one method is a technology in which an R-wave is detected for each section estimated to be one heartbeat from electrocardiographic waveform data that represent periodic fluctuations, and an R-R interval representing an interval from each R-wave to a subsequent R-wave is calculated. Waveform data for one heartbeat is then generated by superposing the waveform data based on each R-R interval and performing weighted-averaging thereon, and representative waveform data is generated by multiplying the generated waveform data by a window function such as a Hanning window. Mutual-correlation processing is then performed on the generated representative waveform data and the electrocardiographic waveform data, and an R-R interval is calculated from the mutual-correlation processed data, and based on the R-R interval, the heart rate can be calculated.

Furthermore, as another method, disclosed is a technology in which biological information by heartbeats of a user is acquired, and after the body movement of the user is finished, waveform data for one heartbeat to be used as a template is generated based on the biological information. The waveform data as the template here is generated by selecting the first piece of waveform data out of a plurality of pieces of waveform data for one heartbeat as a subject of comparison, calculating the degree of similarity with respect to the other pieces of waveform data, and averaging the waveform data of the waveforms of high degree of similarity. A correlation coefficient is then calculated between the template generated and the waveform data of the biological information, and a peak for each one-heartbeat time is identified from the correlation coefficient. Based on the time interval of the peaks, the heart rate can be calculated.

Conventional technologies are described in Japanese Laid-open Patent Publication No. 2004-089314, Japanese Laid-open Patent Publication No. 2005-027944, Japanese Laid-open Patent Publication No. 2006-271731, and Japanese Laid-open Patent Publication No. 2006-262973, for example.

The conventional technologies can, however, fail to generate highly accurate representative waveform data. More specifically, in the one method, while the representative waveform data is generated by weighted-averaging the respective pieces of waveform data, the waveform data used in weighted-averaging are randomly selected. Consequently, this method can fail to generate highly accurate representative waveform data. In the other method, while a template corresponding to the representative waveform data is generated by averaging the waveforms of a high degree of similarity, the respective pieces of waveform data used in averaging are also randomly selected. Consequently, the other method can also fail to generate highly accurate representative waveform data.

### SUMMARY

According to an aspect of an embodiment, an information processing apparatus includes a signal dividing module, a calculating module and a waveform selecting module. The signal dividing module divides a biosignal into waveforms of a fixed interval. The calculating module calculates a plurality of waveform intervals indicative of an interval between adjacent waveforms and calculates an average value of the calculated waveform intervals for each of the waveforms of the fixed interval divided by the signal dividing module. the waveform selecting module selects a plurality of waveforms of the fixed interval corresponding to average values indicating near a maximum value of frequency of average values using the average value of the waveform intervals calculated for each of the waveforms of the fixed interval by the calculating module.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a functional block diagram illustrating the configuration of a handheld terminal according to a first embodiment;

FIG. 2 is a chart illustrating an example of a heartbeat waveform divided by one-minute intervals;

FIG. 3A is a chart for explaining the relation of a time constant and a stable section in a situation of sitting after walking;

FIG. 3B is a chart for explaining the relation of the time constant and the stable section in a situation of sitting after running;

FIG. 3C is a chart for explaining the relation of the time constant and the stable section in a situation of sitting after walking;

FIG. 4 is a chart illustrating an example of a waveform after an electrocardiographic signal is processed with a digital high-pass filter;

FIG. 5A is a chart illustrating an example of a histogram of R-R intervals when activity is sleeping;

FIG. 5B is a chart illustrating an example of the histogram of R-R intervals when the activity is sitting;

FIG. 5C is a chart illustrating an example of the histogram of R-R intervals when the activity is walking;

FIG. 6 is a diagram illustrating a cutout example of a waveform in a unit of one period;

FIG. 7A is a chart illustrating the transition of R-R intervals when sitting after running;

FIG. 7B is a chart illustrating an example of the histogram of R-R intervals when one-minute heartbeat waveforms are extracted from a transition section;

FIG. 7C is a chart illustrating an example of the histogram of R-R intervals when one-minute heartbeat waveforms are extracted from a stable section;

FIG. 8 is a chart illustrating a situation in which candidate waveforms are randomly selected;

FIG. 9 is a diagram illustrating a situation in which candidate waveforms are selected by a process performed by a candidate-waveform selecting processor 140;

FIG. 10 is a flowchart illustrating a representative-waveform generating process performed in the first embodiment;

FIG. 11 is a functional block diagram illustrating the configuration of a handheld terminal according to a second embodiment;

FIG. 12 is a diagram illustrating a concept when independent component analysis is performed on a plurality of one-minute heartbeat waveforms;

FIG. 13 is a diagram illustrating an example of a result of fast Fourier transformation performed on the heartbeat waveforms on which ICA has been performed; and

FIG. 14 is a block diagram illustrating an example of a computer that executes a representative-waveform generating program.

### DESCRIPTION OF EMBODIMENTS

Preferred embodiments of the present invention will be explained with reference to accompanying drawings. The invention is, however, not intended to be restricted by the embodiments. The embodiments can be combined as appropriate within a range not making the content of process inconsistent. In the following description, exemplified is a situation to which the invention is applied with a biosignal as an electrocardiographic signal and the information processing apparatus as a handheld terminal.

### [a] First Embodiment

### Configuration of Handheld Terminal in First Embodiment

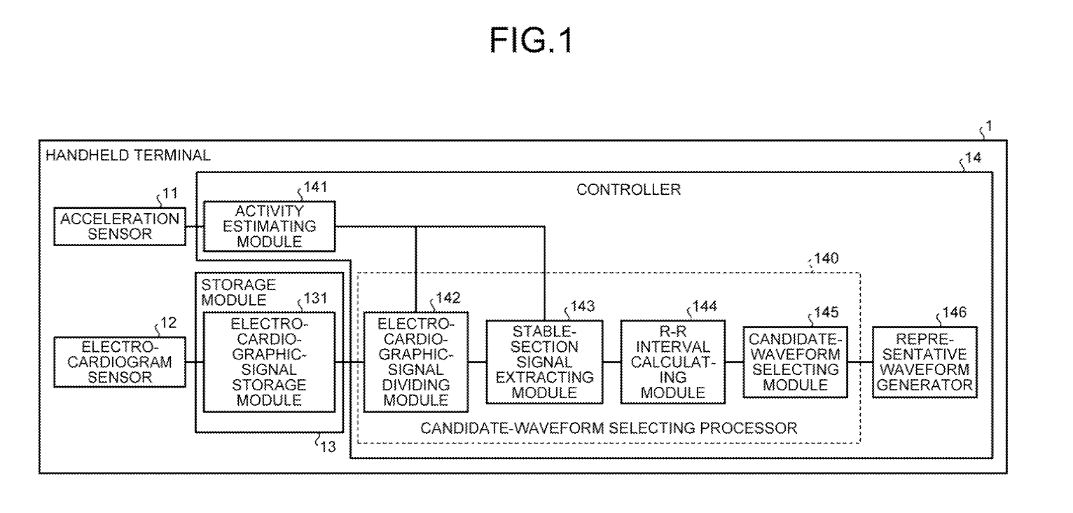

FIG. 1 is a functional block diagram illustrating the configuration of a handheld terminal according to a first embodiment. As illustrated in FIG. 1, a handheld terminal 1 includes an acceleration sensor 11, an electrocardiogram sensor 12, a storage module 13, and a controller 14. The handheld terminal 1 is a device attachable to a body, and is a mobile computer and a cellular phone, as one example.

The acceleration sensor 11 is a sensor that detects acceleration in three axial directions orthogonal to one another. The acceleration sensor 11 is used to analyze the activity of a user attached with the handheld terminal 1, for example. To analyze the activity of the user in more detail, not limiting to only the acceleration sensor 11, combined may be a gyro sensor that detects angular velocity, a geomagnetic sensor that detects terrestrial magnetism, and a GPS sensor that detects the current location (latitude, longitude) of the user, other than the acceleration sensor 11.

The electrocardiogram sensor 12 is a sensor to detect an electrocardiographic signal. The electrocardiogram sensor 12 detects electromotive forces of the heart as the electrocardiographic signal. The electromotive forces of the heart are bioelectric phenomena with a voltage of several millivolts (mV), a frequency of 0.1 to 200 Hertz (Hz), and an impedance of 1 to 20 kilo-ohms (kΩ). The electromotive forces of the heart are normally detected by amplifying a potential difference between electrodes arranged on the surface of the body using an electrical circuit. Consequently, the electrocardiogram sensor 12 includes two or more electrodes, an electrical circuit that detects a potential difference and amplifies the potential difference, a digital signal circuit that converts and records an analog signal into a digital signal at appropriate sampling intervals, and others. The electrocardiogram sensor 12 then outputs digital values of the electrocardiographic signal.

The storage module 13 corresponds to a storage device such as a non-volatile semiconductor memory device of a flash memory and a ferroelectric random access memory (FRAM, registered trademark). The storage module 13 includes an electrocardiographic-signal storage module 131.

The electrocardiographic-signal storage module 131 stores therein data of the electrocardiographic signal. For example, the electrocardiographic-signal storage module 131 stores therein the digital values of the electrocardiographic signal as electrocardiographic data being associated with the measured time. While the data of the electrocardiographic signal are stored for 24 hours, for example, it is not limited to this.

The controller 14 includes an internal memory to store therein programs that define procedures of various processes and control data, and executes the various processes with the foregoing. The controller 14 corresponds to an integrated circuit such as an application specific integrated circuit (ASIC) and a field programmable gate array (FPGA) or an electronic circuit such as a central processing unit (CPU) and a micro processing unit (MPU), for example. The controller 14 further includes a candidate-waveform selecting processor 140, an activity estimating module 141, and a representative waveform generator 146. The candidate-waveform selecting processor 140 includes an electrocardiographic-signal dividing module 142, a stable-section signal extracting module 143, an R-R interval calculating module 144, and a candidate-waveform selecting module 145.

The activity estimating module 141 estimates the activity of the user attached with the handheld terminal 1 from the acceleration detected by the acceleration sensor 11, for example. The activity estimating module 141 further outputs activity information representing the activity estimated, the start time of the activity, and the end time of the activity.

As for the method to estimate activities of a human, a variety of methods have already been developed. For example, in the literature of “Human Activity Sensors Consortium (HASC) Challenge 2010: Construction of Wearable Accelerometer Sensor Corpus for Activity Recognition”, it is stated that the recognition of a total of six types of activities of standstill, walking, jogging, skipping, going up the stairs, and going down the stairs has been attained. Furthermore, in the literature of “Challenges on Activity Recognition Techniques using Wearable Sensors”, presented are the examples of recognizing walking, bicycle riding, and standstill (coming to a stop) by acceleration sensors. The types of activities estimated by the activity estimating module 141 and the method of estimating activities performed by the activity estimating module 141 are not specifically limited.

The electrocardiographic-signal dividing module 142 divides the electrocardiographic signal into waveforms of a fixed time interval. The electrocardiographic-signal dividing module 142 uses the electrocardiographic data stored in the electrocardiographic-signal storage module 131 and divides a heartbeat waveform of the electrocardiographic signal by an appropriate time interval, for example. While the fixed time interval is defined as one-minute interval, for example, it may be two-minute interval or three-minute interval. In the following description, the fixed time interval is exemplified as one-minute interval.

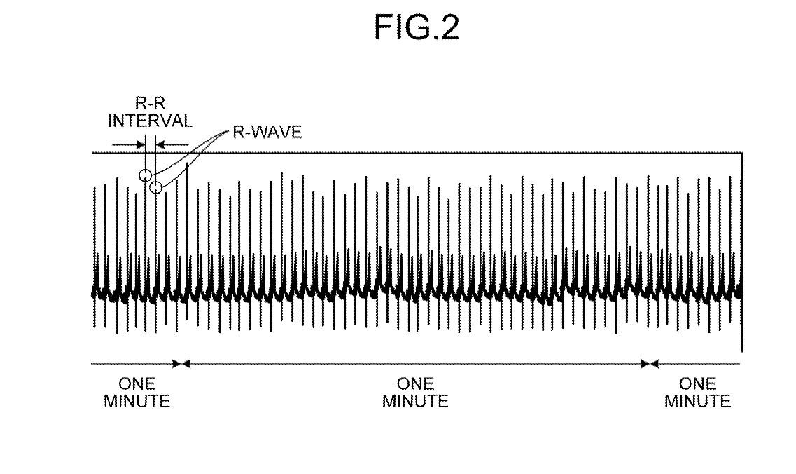

An example of dividing a heartbeat waveform by a fixed time interval will now be described with reference to FIG. 2. FIG. 2 is a chart illustrating an example of a heartbeat waveform divided by one-minute intervals. As illustrated in FIG. 2, the heartbeat waveform of an electrocardiographic signal is represented on the graphic chart. The electrocardiographic-signal dividing module 142 divides the heartbeat waveform by one-minute interval. Pointed peaks of the graphic chart are R-waves. The R-wave is a wave produced when a heart contracts, representing that the force of electric current flow is strong. An interval from an R-wave representing a peak to an R-wave representing a subsequent peak (R-R interval) corresponds to one period of heartbeat. In the following description, the waveform divided by one-minute interval is referred to as a one-minute heartbeat waveform, and is defined as one example of the waveform divided by a fixed time interval.

Referring back to FIG. 1, the electrocardiographic-signal dividing module 142 associates each of the one-minute heartbeat waveforms with activity. The electrocardiographic-signal dividing module 142 associates each of the one-minute heartbeat waveforms with activity information based on the start time and end time of the respective activities obtained by the activity estimating module 141, for example.

The stable-section signal extracting module 143 extracts a plurality of one-minute heartbeat waveforms for each activity. When extracting one-minute heartbeat waveforms for a given activity, the stable-section signal extracting module 143 extracts the one-minute heartbeat waveforms from a stable section that is a section after the elapse of a time period (referred to as a time constant) from the time when the activity is changed to the given activity until the waveforms stabilize. This is to extract the one-minute heartbeat waveforms after the heart rate stabilizes for the given activity. For example, when sitting after running, the shape of heartbeat waveform varies until the heart rate stabilizes, and thus uniform waveforms for the activity of sitting are not extracted. Consequently, the stable-section signal extracting module 143 extracts the one-minute heartbeat waveforms from the stable section after the elapse of a time constant. The time constant is defined in advance from the combination of previous and subsequent activities. The method of calculating the time constant will be described later.

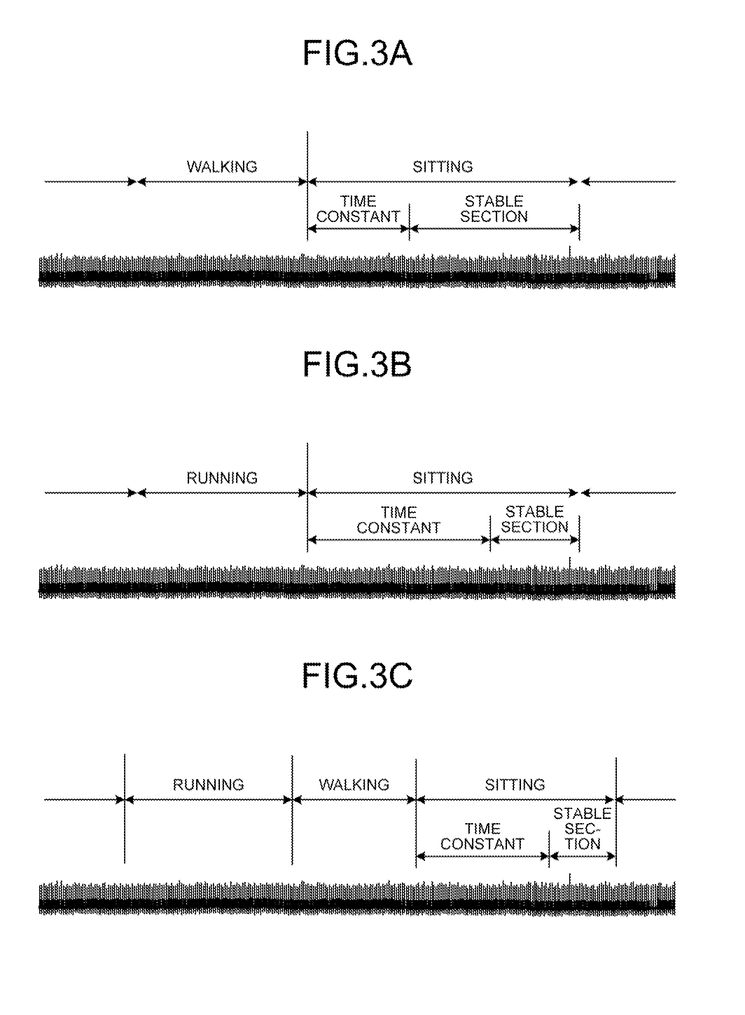

The relation of the time constant and the stable section will now be described with reference to FIGS. 3A, 3B, and 3C. FIG. 3A is a chart for explaining the relation of the time constant and the stable section in a situation of sitting after walking. FIG. 3B is a chart for explaining the relation of the time constant and the stable section in a situation of sitting after running. FIG. 3C is a chart for explaining the relation of the time constant and the stable section in a situation of sitting after walking.

As illustrated in FIG. 3A, the heartbeat waveform in a situation of sitting after walking is represented. When the situation in which the previous activity is walking and the situation in which the previous activity is running are compared, the time constant from the time when sitting is started until the heart rate stabilizes is shorter when the previous activity is walking. The stable-section signal extracting module 143 thus extracts the one-minute heartbeat waveforms included in the stable section, using a time constant for the situation in which the previous activity is walking and the subsequent activity is sitting, after the elapse of the time constant from the time when walking is changed to sitting.

As illustrated in FIG. 3B, the heartbeat waveform in a situation of sitting after running is represented. When the situation in which the previous activity is walking and the situation in which the previous activity is running are compared, the time constant from the time when sitting is started until the heart rate stabilizes is longer when the previous activity is running. The stable-section signal extracting module 143 thus extracts the one-minute heartbeat waveforms included in the stable section, using a time constant for the situation in which the previous activity is running and the subsequent activity is sitting, after the elapse of the time constant from the time when running is changed to sitting.

The time constant has been exemplified to be defined by the combination of a focused activity (sitting, here) and the one previous activity. The time constant is, however, not limited to this, and may be defined by the combination of the focused activity, one previous activity, and a further previous activity. In FIG. 3C, the time constant is defined by sitting representing the focused activity, walking representing the one previous activity, and running representing the further previous activity. The stable-section signal extracting module 143 then extracts, using a predetermined time constant, the one-minute heartbeat waveforms included in the stable section after the elapse of the time constant from the time when walking is changed to sitting.

The R-R interval calculating module 144 calculates a plurality of R-R intervals for each of the one-minute heartbeat waveforms extracted from the stable section by the stable-section signal extracting module 143. The R-R interval calculating module 144 applies the signal processing of a high-pass filter, which passes only high frequencies higher than a given frequency, to the one-minute heartbeat waveforms extracted to detect R-waves. The R-R interval calculating module 144 then calculates an interval between two adjacent R-waves, i.e., the R-R interval, for the R-waves detected.

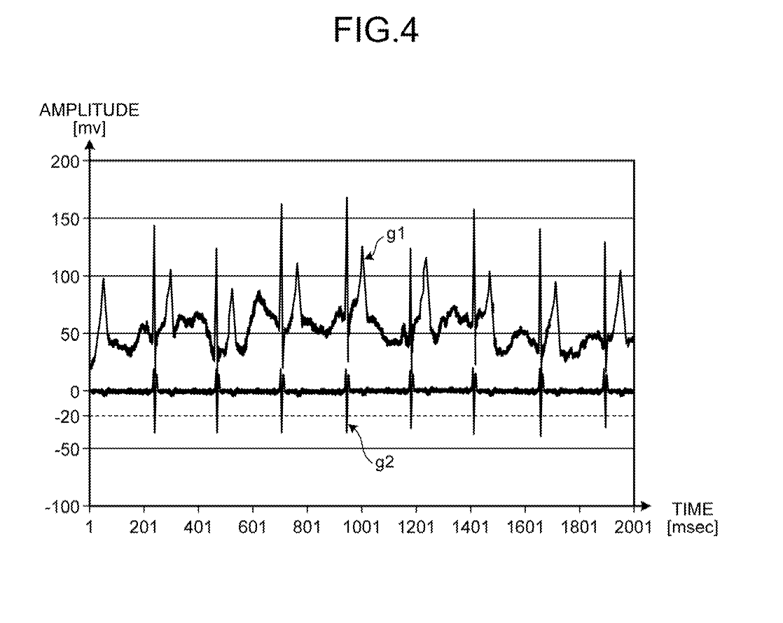

FIG. 4 is a chart illustrating an example of a waveform after an electrocardiographic signal is processed with a digital high-pass filter. As illustrated in FIG. 4, in the graphic chart of the waveform of the electrocardiographic signal, the X axis represents time and the Y axis represents amplitude. A waveform g2, which is a waveform after an electrocardiographic signal g1 is processed with the high-pass filter, is equal to or lower than −20 at the positions of R-waves. The R-R interval calculating module 144 thus detects, with a threshold of −20 here, the time when the signal applied with the high-pass filter is equal to or lower than −20, i.e., the time of an R-wave. The R-R interval calculating module 144 then calculates the R-R intervals using the detected time of R-waves.

Referring back to FIG. 1, the R-R interval calculating module 144 calculates an average value of R-R intervals for each one-minute heartbeat waveform using a plurality of R-R intervals calculated for each one-minute heartbeat waveform. The R-R interval calculating module 144 further calculates standard deviation for each one-minute heartbeat waveform using the R-R intervals and the average value calculated for each one-minute heartbeat waveform.

The candidate-waveform selecting module 145 selects a plurality of one-minute heartbeat waveforms that are the one-minute heartbeat waveforms indicating near a maximum value of frequency of the average value of the R-R intervals calculated for each one-minute heartbeat waveform and indicating low values of standard deviation. In other words, the candidate-waveform selecting module 145 generates candidate waveforms to be used when a representative waveform is generated by the representative waveform generator 146, which will be described later. The representative waveform is a waveform for a typical one period, more specifically, for a one heartbeat, and is generated for each activity.

The candidate-waveform selecting module 145 sorts the one-minute heartbeat waveforms used by the R-R interval calculating module 144 for each activity, for example. In other words, the candidate-waveform selecting module 145 sorts the one-minute heartbeat waveforms extracted from the stable section of each activity by the stable-section signal extracting module 143 for each activity. The candidate-waveform selecting module 145 then generates a histogram of R-R intervals according to activity with the abscissa axis as average value of R-R intervals and the ordinate axis as frequency, for the one-minute heartbeat waveforms sorted for each activity. The reason for generating the histogram of R-R intervals according to activity is because the heart rate normally differs by the type of activity, and thus the position representing the maximum value of frequency of histogram (average value of R-R intervals) also differs by the type of activity. The candidate-waveform selecting module 145 then selects, for each activity, an appropriate number of one-minute heartbeat waveforms corresponding to the average value of R-R intervals indicating near the maximum value of frequency. In other words, the candidate-waveform selecting module 145 generates candidate waveforms for each activity.

As one example, assuming that the maximum value of R-R interval is tₘₐₓ, and the R-R interval of the i-th one-minute heartbeat waveform is tᵢ, the candidate-waveform selecting module 145 selects the one-minute heartbeat waveforms for which the absolute value of the difference in R-R intervals is equal to or smaller than a predetermined threshold t_{TH} as expressed in the following Expression (1).

|tᵢ−tₘₐₓ|≦t_{TH} (1)

The candidate-waveform selecting module 145 further selects an appropriate number of one-minute heartbeat waveforms in ascending order of standard deviation of R-R interval out of the one-minute heartbeat waveforms selected.
Consequently, the candidate-waveform selecting module 145 selects the waveforms for which the average value of R-R intervals is nearly identical and the variations in R-R intervals for one heartbeat period are small, and thus the candidates of uniform heartbeat waveforms can be selected according to activity.

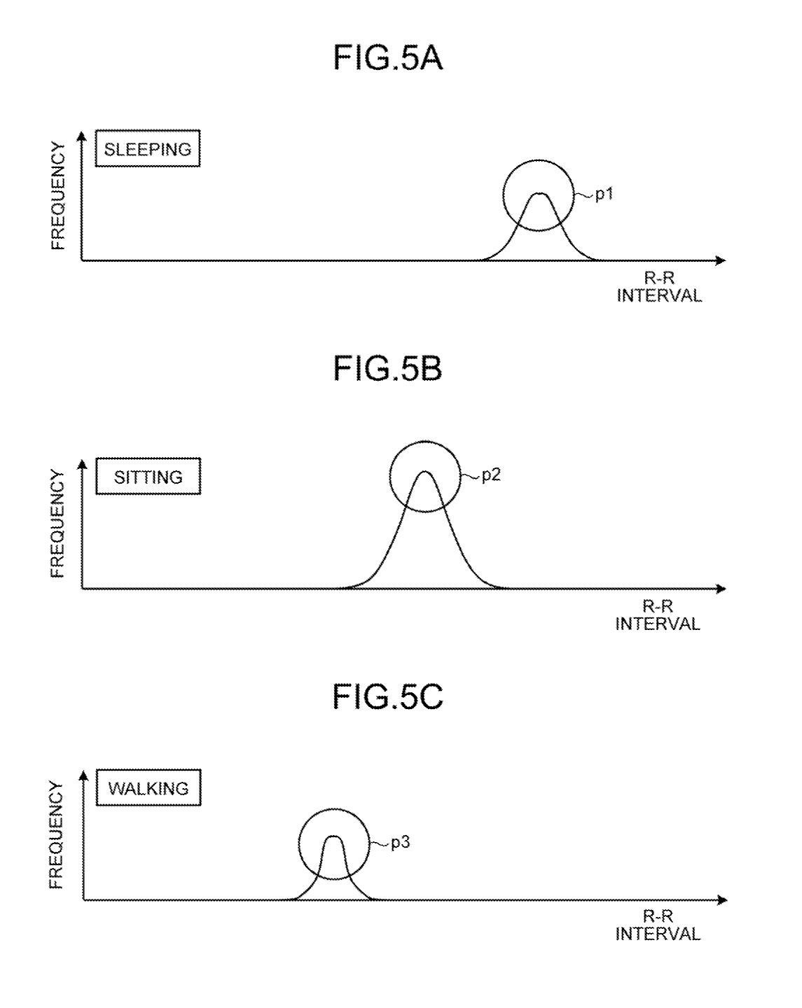

The examples of histogram of R-R intervals corresponding to each activity will now be described with reference to FIGS. 5A, 5B, and 5C. FIG. 5A is a chart illustrating an example of the histogram of R-R intervals when the activity is sleeping. FIG. 5B is a chart illustrating an example of the histogram of R-R intervals when the activity is sitting. FIG. 5C is a chart illustrating an example of the histogram of R-R intervals when the activity is walking.

As illustrated in FIG. 5A, when the activity is sleeping, the position indicating the maximum value of frequency (average value of R-R intervals) is larger, as compared with when the activity is sitting as illustrated in FIG. 5B and when the activity is walking as illustrated in FIG. 5C. This is because the heart rate normally lowers while sleeping than while sitting or walking.

As illustrated in FIG. 5B, when the activity is sitting, the position indicating the maximum value of frequency (average value of R-R intervals) is smaller as compared with when the activity is sleeping as illustrated in FIG. 5A, and is larger as compared with when the activity is walking as illustrated in FIG. 5C. This is because the heart rate normally increases while sitting than sleeping, and the heart rate lowers while sitting than walking.

As illustrated in FIG. 5C, when the activity is walking, the position indicating the maximum value of frequency (average value of R-R intervals) is smaller, as compared with when the activity is sleeping as illustrated in FIG. 5A and when the activity is sitting as illustrated in FIG. 5B. This is because the heart rate normally increases while walking than while sleeping or sitting.

Consequently, the candidate-waveform selecting module 145 assumes that the one-minute heartbeat waveforms corresponding to average values of R-R intervals near the maximum values p1, p2, and p3 of frequency correspond to the representative one-minute heartbeat waveforms of the respective activities. The candidate-waveform selecting module 145 then selects an appropriate number of one-minute heartbeat waveforms near the maximum value of frequency for each activity and defines them as the candidate waveforms for the respective activities.

Referring back to FIG. 1, the representative waveform generator 146 generates a representative waveform in a unit of one period according to activity.

For example, the representative waveform generator 146 cuts out waveforms in a unit of one period from the one-minute heartbeat waveforms obtained by the candidate-waveform selecting module 145 using the position of the R-wave according to activity. At this time, the representative waveform generator 146 selects, as an end point to cut out, the position at which the gradient of the one-minute heartbeat waveform is small. The representative waveform generator 146 selects the end point to cut out such that the waveform is divided in a ratio of 4:6 with respect to the peak position of the R-wave as a reference, as one example.

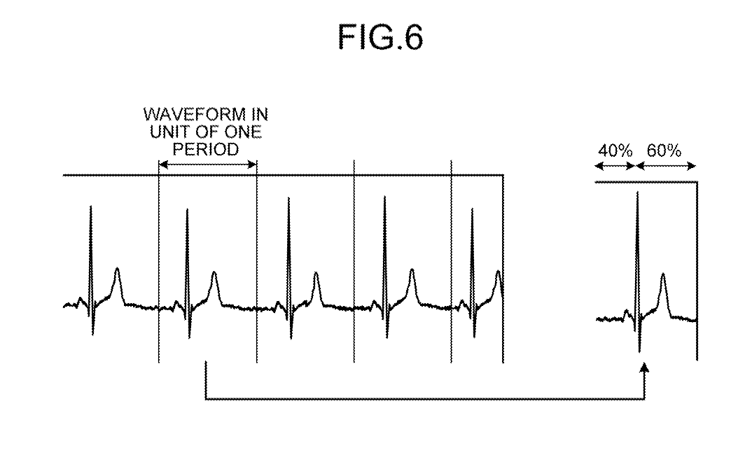

The cutout of a waveform in a unit of one period will now be described with reference to FIG. 6. FIG. 6 is a diagram illustrating a cutout example of a waveform in a unit of one period. As illustrated in FIG. 6, the representative waveform generator 146 cuts out a waveform in a unit of one period such that the waveform is divided into a first half of 40 percent and a second half of 60 percent with respect to the peak position of R-wave of a one-minute heartbeat waveform as a reference. While the end point to cut out is described to cut out to divide the waveform in a ratio of 4:6 with respect to the peak position of R-wave as the reference, it is not limited to this. The end point to cut out may be defined to cut out to divide the waveform in a ratio of 3:7 with respect to the peak position of R-wave as the reference, or may be defined to cut out to divide the waveform in a ratio of 5:5, in which case it only needs to cut out the waveform at the same rate with respect to the peak position of R-wave as the reference.

Referring back to FIG. 1, the representative waveform generator 146 averages all of the waveforms cut out in a unit of one period to generate a tentative representative waveform. The following process of the representative waveform generator 146 is performed according to activity. As one example, it is assumed that P pieces of candidate waveforms (one-minute heartbeat waveforms) for an activity are selected. It is further assumed that N pieces of waveforms in a unit of one period are included in a one-minute heartbeat waveform. Furthermore, the waveform in a unit of one period is assumed to be given by M pieces of sampling points. Under such definition, when the value of the j-th waveform in a unit of one period at the time of the k-th sampling is expressed as S^{(j)}(tₖ), a tentative representative waveform S′(tₖ) is expressed by the following expression:

$$\begin{matrix}\begin{matrix}\begin{matrix}{S}^{′} ( {t}_{i} ) = \frac{1}{P \times N} {\sum }_{j = 1}^{P \times N} {S}^{( j )} ( {t}_{1} ) \\ ⋮ \\ {S}^{′} ( {t}_{k} ) = \frac{1}{P \times N} {\sum }_{j = 1}^{P \times N} {S}^{( j )} ( {t}_{k} )\end{matrix} \\ ⋮\end{matrix} \\ {S}^{′} ( {t}_{p} ) = \frac{1}{P \times N} {\sum }_{j = 1}^{P \times N} {S}^{( j )} ( {t}_{p} )\end{matrix} .$$

The representative waveform generator 146 calculates the degree of similarity between the tentative representative waveform and all of the waveforms cut out in a unit of one period, and selects an appropriate number of waveforms in descending order of degree of similarity. As one example, the degree of similarity between the waveforms is expressed as a sum of squares of the difference between the waveforms as expressed in the following Expression (2).

$${L}^{( j )} = {\sum }_{k = 1}^{M} {\{ {S}^{( j )} ( {t}_{k} ) - {S}^{′} ( {t}_{k} ) \}}^{2} \qquad ( 2 )$$

The representative waveform generator 146 selects the waveforms in a unit of one period that satisfy the degree of similarity L^{(j)} being equal to or smaller than a threshold L_{TH}, and from the averaging of the waveforms in a unit of one period selected, generates a final representative waveform. As one example, the generation of representative waveform is expressed as in the following Expression (3).

$$S ( {t}_{k} ) = \frac{1}{{N}_{SEL}} {\sum }_{j ∈ {j}_{SEL}}^{ } {S}^{( j )} ( {t}_{k} ) \qquad ( 3 )$$

where j_{SEL} represents the value of j that satisfies L^{(j)}≦L_{TH}, and N_{SEL} represents the total number of waveforms in a unit of one period that satisfy L^{(j)}≦L_{TH}. More specifically, S(tₖ) is generated as the final representative waveform.

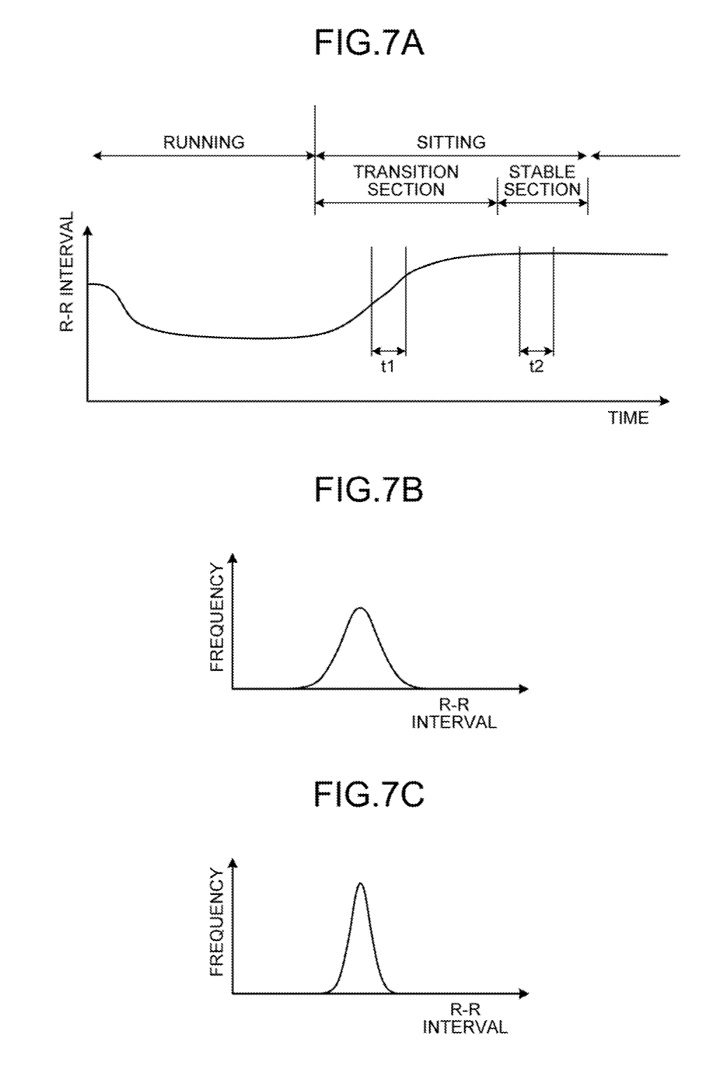

Next, the method of calculating a time constant used by the stable-section signal extracting module 143 will be described with reference to FIGS. 7A, 7B, and 7C. In FIGS. 7A, 7B, and 7C, a situation of sitting after running is explained as one example. FIG. 7A is a chart illustrating the transition of R-R intervals when sitting after running. FIG. 78 is a chart illustrating an example of the histogram of R-R intervals when one-minute heartbeat waveforms are extracted from a transition section. FIG. 7C is a chart illustrating an example of the histogram of R-R intervals when one-minute heartbeat waveforms are extracted from a stable section.

As illustrated in FIG. 7A, in the graphic chart of the transition of R-R interval, the X axis is defined as time and the Y axis is defined as R-R interval. When sitting after running, the R-R interval in running is approximately smaller than that after sitting. The R-R interval after sitting gradually increases in the transition section representing a period of unstable heartbeat waveforms. The R-R interval after sitting thereafter reaches an approximately stable value in the stable section representing a period of stable heartbeat waveforms after going through the transition section.

As illustrated in FIGS. 7B and 7C, in the histograms of the R-R intervals, the X axis is defined as average value of R-R intervals and the Y axis is defined as frequency. FIG. 7B illustrates the histogram of R-R intervals of the one-minute heartbeat waveforms extracted from a time period t1 in the transition section. FIG. 7C illustrates the histogram of R-R intervals of the one-minute heartbeat waveforms extracted from a time period t2 in the stable section. In the histogram in FIG. 7B, because the one-minute heartbeat waveforms extracted from the time period t1 in the transition section are used, the variations in the value of R-R intervals are large, and thus the distribution of R-R intervals has a shape with a broad base. In the histogram in FIG. 7C, because the one-minute heartbeat waveforms extracted from the time period t2 in the stable section are used, the variations in the value of R-R intervals are small, and thus the distribution of R-R intervals has a shape with a narrow base. In other words, in the histogram in FIG. 7B, the standard deviation of R-R intervals is higher as compared with that of the histogram in FIG. 7C. On the other hand, in the histogram in FIG. 7C, the standard deviation of R-R intervals is lower as compared with that of the histogram in FIG. 7B.

Consequently, in the time-constant calculating method, the transition to the stable section is detected by the standard deviation in the transition section reaching a value equal to or smaller than the threshold, and an elapsed time from the time when running is changed to sitting until the time when the transition is detected as the time constant is calculated, for example. In the time-constant calculating method, the standard deviation of R-R intervals for each one-minute heartbeat waveform is acquired which is obtainable using the activity estimating module 141, the electrocardiographic-signal dividing module 142, and the R-R interval calculating module 144, for example. In the time-constant calculating method, the time of the standard deviation reaching a value equal to or smaller than the threshold from the time when running is changed to sitting is detected using the standard deviation of R-R intervals of the one-minute heartbeat waveforms acquired. In the time-constant calculating method, the time elapsed from the time when running is changed to sitting until the detected time is then calculated as the time constant.

The time constant only needs to be calculated before a representative-waveform generating process is performed to generate a representative waveform, for example, in a trial period to calculate the time constant. Furthermore, while the situation of sitting after running has been described here, the time constant in the combination of a focused activity and one previous activity can be calculated in the same manner from the combination of previous and subsequent activities.

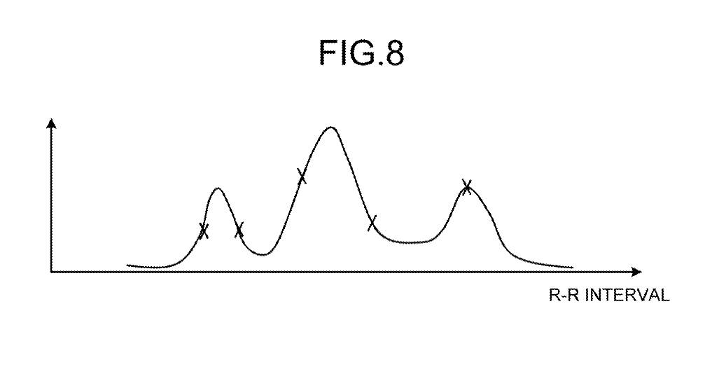

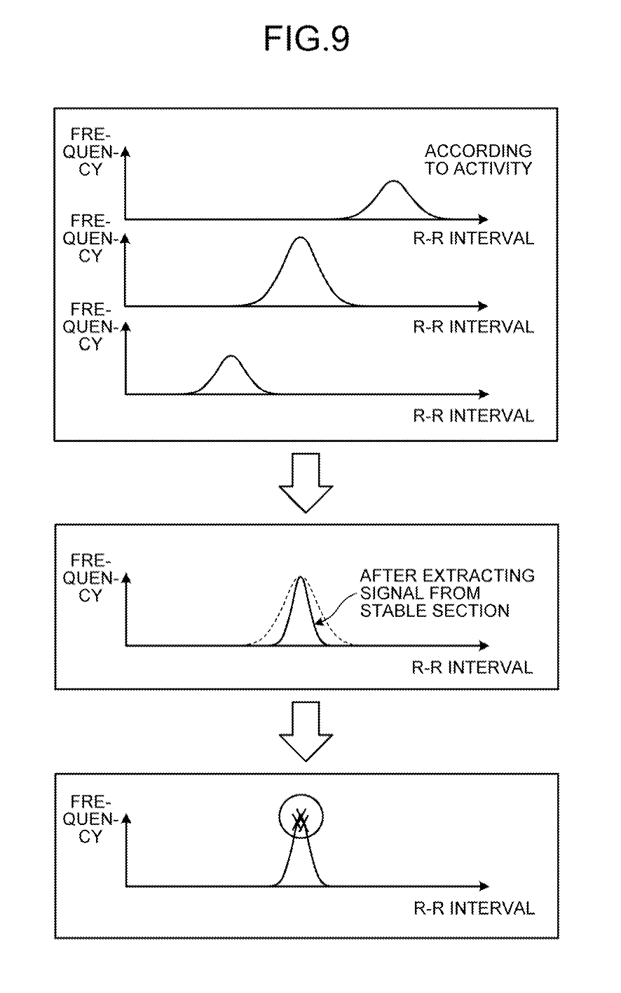

The process of the candidate-waveform selecting processor 140 will now be described conceptually with reference to FIGS. 8 and 9. FIG. 8 is a chart illustrating a situation in which candidate waveforms are randomly selected. FIG. 9 is a diagram illustrating a situation in which candidate waveforms are selected by the process performed by the candidate-waveform selecting processor 140. As illustrated in FIG. 8, when the candidate waveforms are randomly selected, the R-R intervals of the respective candidate waveforms selected vary widely, and thus the candidate waveform calculated by averaging, for example, will result in large distortion.

In contrast, as illustrated in FIG. 9, the electrocardiographic-signal dividing module 142 divides an electrocardiographic signal into one-minute heartbeat waveforms and associates the divided one-minute heartbeat waveforms with activity, thereby enabling the candidate-waveform selecting module 145 to generate a histogram of R-R intervals according to activity. The stable-section signal extracting module 143 then extracts the one-minute heartbeat waveforms from the stable section according to the activity focused, thereby enabling the candidate-waveform selecting module 145 to generate a histogram of small variations in the R-R intervals according to activity. Furthermore, the candidate-waveform selecting module 145 selects one-minute heartbeat waveforms of low standard deviations near the maximum value of histogram of R-R intervals according to activity, thereby enabling it to select uniform one-minute heartbeat waveforms according to activity. In other words, the candidate-waveform selecting module 145 can select uniform candidate waveforms of the focused activity. Consequently, the representative waveform generator 146 can generate a typical and uniform representative waveform of a focused activity in a unit of one period.

Procedure of Representative-Waveform Generating Process

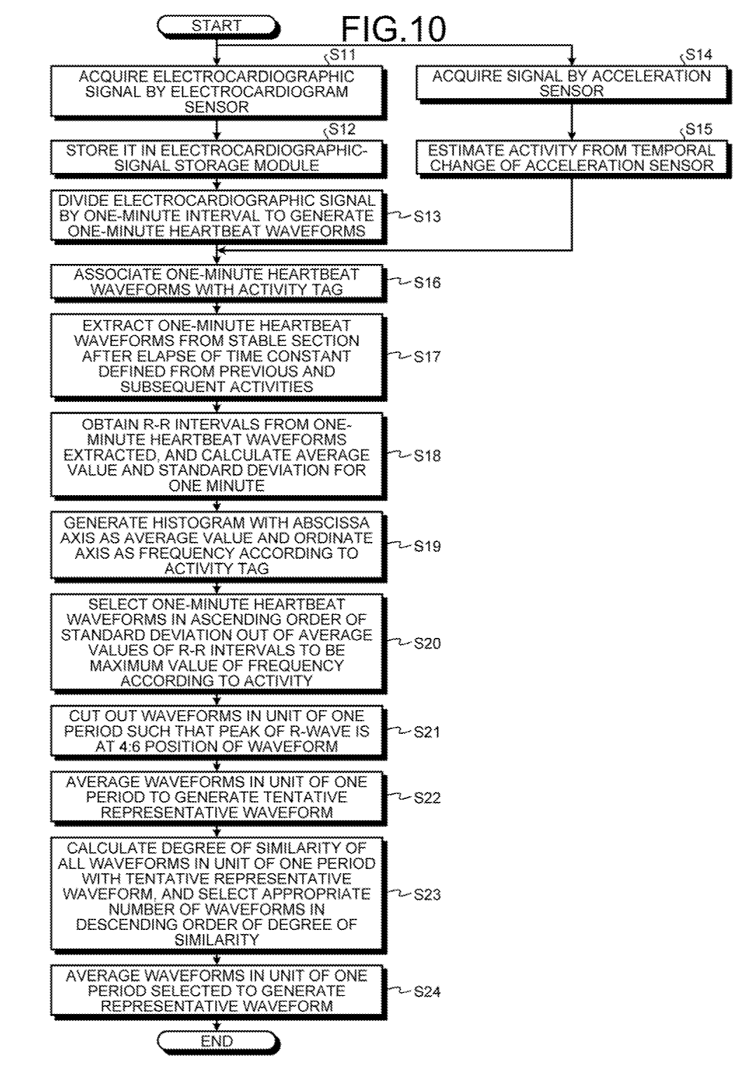

Next, the procedure of representative-waveform generating process will be described with reference to FIG. 10. FIG. 10 is a flowchart illustrating the representative-waveform generating process performed in the first embodiment. It is assumed that the user is attached with the handheld terminal 1 and the electrodes of the electrocardiogram sensor 12 are disposed on the body surface.

The handheld terminal 1 first acquires an electrocardiographic signal by the electrocardiogram sensor 12 (Step S11), and stores the data of the electrocardiographic signal acquired in the electrocardiographic-signal storage module 131 (Step S12). For example, the handheld terminal 1 associates the digital values of the electrocardiographic signal with the measured time and stores them in the electrocardiographic-signal storage module 131. The electrocardiographic-signal dividing module 142 then divides the electrocardiographic signal by one-minute interval using the data of the electrocardiographic signal stored in the electrocardiographic-signal storage module 131 to generate one-minute heartbeat waveforms (Step S13).

Independently of the electrocardiogram sensor 12, the handheld terminal 1 acquires a signal concerning acceleration by the acceleration sensor 11 (Step S14). The activity estimating module 141 then estimates activity from the temporal changes of the acceleration sensor 11 (Step S15).

The electrocardiographic-signal dividing module 142 then associates the one-minute heartbeat waveforms generated with an activity tag (Step S16). The electrocardiographic-signal dividing module 142 associates the one-minute heartbeat waveform with activity using the measurement start time and measurement end time of the one-minute heartbeat waveform and the start time and end time of the activity estimated by the activity estimating module 141, and associates the activity tag of the associated activity with the one-minute heartbeat waveform, for example.

Subsequently, the stable-section signal extracting module 143 extracts a plurality of one-minute heartbeat waveforms from the stable section after the elapse of a time constant defined from the previous and subsequent activities (Step S17). The stable-section signal extracting module 143 extracts, when the activity tag represents sitting, a plurality of one-minute heartbeat waveforms from the stable section after the elapse of the time constant from the time when the activity tag is changed to sitting, for example. The time constant is determined in advance from the combination of the activity before sitting and the sitting.

The R-R interval calculating module 144 then obtains a plurality of R-R intervals from the one-minute heartbeat waveform extracted, and calculates an average value and standard deviation of the R-R intervals for one minute (Step S18). More specifically, the R-R interval calculating module 144 calculates the average value and the standard deviation of R-R intervals for one minute for the respective one-minute heartbeat waveforms extracted by the stable-section signal extracting module 143 for each activity tag.

The candidate-waveform selecting module 145 then generates a histogram of R-R intervals with the abscissa axis as the average value of R-R intervals of one-minute heartbeat waveform and the ordinate axis as the frequency of the one-minute heartbeat waveform according to an activity tag (Step S19). The candidate-waveform selecting module 145 then selects, according to the activity tag, an appropriate number of one-minute heartbeat waveforms in ascending order of standard deviation out of the one-minute heartbeat waveforms corresponding to the average value of R-R intervals to be the maximum value of frequency (Step S20). More specifically, the candidate-waveform selecting module 145 selects an appropriate number of candidate waveforms for each activity.

The representative waveform generator 146 then generates, according to the activity tag, a representative waveform in the following manner. The representative waveform generator 146 cuts out waveforms in a unit of one period such that the peak of R-wave is positioned in a ratio of 4:6 of the waveform for the respective one-minute heartbeat waveforms selected by the candidate-waveform selecting module 145 (Step S21). The representative waveform generator 146 then averages the waveforms in a unit of one period to generate a tentative representative waveform (Step S22).

Subsequently, the representative waveform generator 146 calculates the degree of similarity between all of the waveforms in a unit of one period and the tentative representative waveform, and selects an appropriate number of waveforms in a unit of one period in descending order of degree of similarity (Step S23). The representative waveform generator 146 selects 10 pieces of the waveforms in a unit of one period, for example. The representative waveform generator 146 then averages the appropriate number of waveforms in a unit of one period selected to generate a representative waveform (Step S24).

The representative waveforms generated in the foregoing manner according to activity are stored in the handheld terminal, or in an external device such as a personal computer and a cloud. Comparing them with electrocardiographic waveforms output in respective daily activities enables abnormalities such as arrhythmia to be detected. Alternatively, when a marathon runner or the like takes a long-term practice, observing the changes in electrocardiographic waveforms in running enables the effects of training to be grasped.

### Advantageous Effects of First Embodiment

In accordance with the first embodiment, the handheld terminal 1 divides a biosignal into one-minute heartbeat waveforms. The handheld terminal 1 then calculates a plurality of R-R intervals for each of the one-minute heartbeat waveforms divided, and calculates an average value of the R-R intervals. Furthermore, the handheld terminal 1 selects a plurality of one-minute heartbeat waveforms corresponding to average values indicating near the maximum value of frequency of average values using the average values of the R-R intervals calculated for the respective one-minute heartbeat waveforms. The handheld terminal 1 with this configuration selecting the one-minute heartbeat waveforms corresponding to the average values near the maximum value of frequency of the average values of R-R intervals enables it to select the one-minute heartbeat waveforms of uniform R-R intervals. As a consequence, the handheld terminal 1 can generate a highly accurate representative waveform for one period using the one-minute heartbeat waveforms selected.

In accordance with the first embodiment, the handheld terminal 1 associates the one-minute heartbeat waveforms with the activity estimated by the activity estimating module 141. The handheld terminal 1 then selects a plurality of one-minute heartbeat waveforms corresponding to a single activity. Because the R-R intervals differ depending on the type of activity, the handheld terminal 1 with this configuration selecting the one-minute heartbeat waveforms corresponding to the activity enables it to select further uniform one-minute heartbeat waveforms.

Furthermore, in accordance with the first embodiment, the handheld terminal 1 extracts the one-minute heartbeat waveforms corresponding to a subsequent activity from the stable section after the elapse of a time period (time constant) from the time when a previous activity is changed to the subsequent activity until the waveforms stabilize. The handheld terminal 1 then calculates a plurality of R-R intervals for each of the one-minute heartbeat waveforms extracted corresponding to the subsequent activity, and calculates an average value of the R-R intervals. The handheld terminal 1 further selects a plurality of one-minute heartbeat waveforms corresponding to average values indicating near the maximum value of frequency of average values using the average values of the R-R intervals calculated for the respective one-minute heartbeat waveforms. The handheld terminal 1 with this configuration extracts the one-minute heartbeat waveforms corresponding to the subsequent activity from the stable section of the subsequent activity, and can select the one-minute heartbeat waveforms of small variations.

In accordance with the first embodiment, the handheld terminal 1 further calculates standard deviation of R-R intervals for each one-minute heartbeat waveform using a plurality of R-R intervals calculated for each one-minute heartbeat waveform. The handheld terminal 1 then selects a plurality of one-minute heartbeat waveforms corresponding to average values indicating near the maximum value of frequency of average values and corresponding to low values of standard deviation out of the calculated standard deviations. The handheld terminal 1 with this configuration is to select the one-minute heartbeat waveforms of small variations in R-R intervals, and can select further uniform one-minute heartbeat waveforms. As a consequence, the handheld terminal 1 can generate a representative waveform of a small distortion using the one-minute heartbeat waveforms selected.

In accordance with the first embodiment, the handheld terminal 1 calculates, using standard deviations of R-R intervals of the one-minute heartbeat waveforms from the time when a previous activity is changed to a subsequent activity, a time period from the time when the previous activity is changed to the subsequent activity until the standard deviation is equal to or smaller than a threshold as a time constant. The handheld terminal 1 with this configuration calculates a time constant using standard deviations of R-R intervals, and can reliably calculate the time period reaching the period of small variations in R-R intervals (stable section) as the time constant.

### [b] Second Embodiment

In the first embodiment, the handheld terminal 1 has been exemplified to select one-minute heartbeat waveforms indicating near the maximum value of frequency of the average value of R-R intervals according to activity out of the one-minute heartbeat waveforms extracted according to activity. The handheld terminal 1 is, however, not limited to this, and to reduce noise, an independent component analysis may further be performed on one-minute heartbeat waveforms selected according to activity.

Thus, in a second embodiment, described is a handheld terminal 1A that further performs independent component analysis on one-minute heartbeat waveforms selected according to activity.

Configuration of Handheld Terminal in Second Embodiment

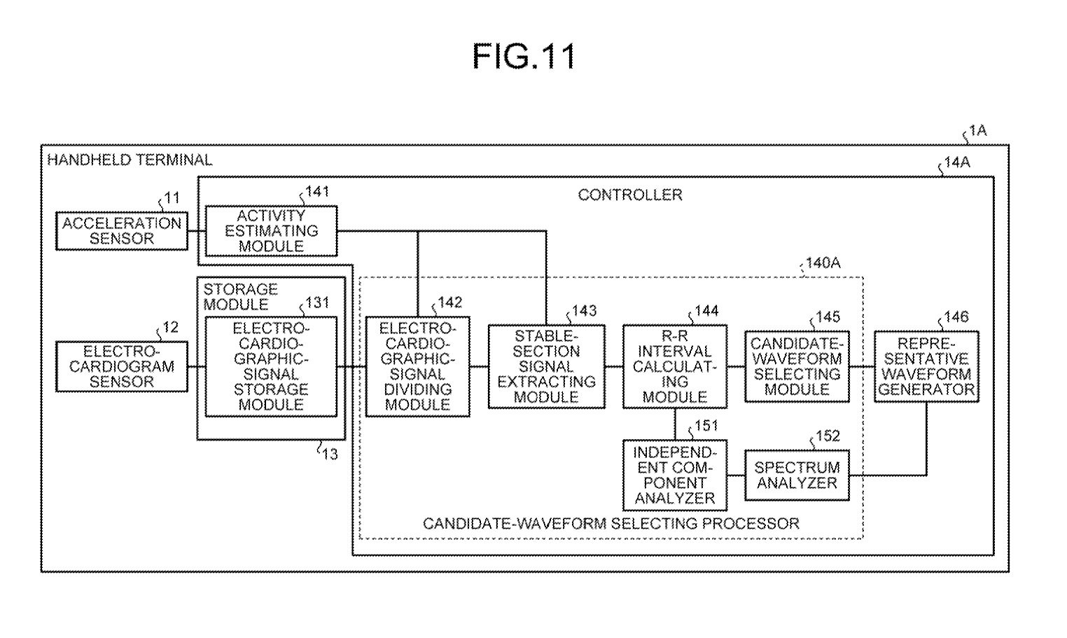

FIG. 11 is a functional block diagram illustrating the configuration of a handheld terminal according to the second embodiment. The same constituents as those of the handheld terminal 1 illustrated in FIG. 1 are indicated by the same reference numerals and symbols, and the redundant explanations of configurations and operations thereof are omitted. The difference between the first embodiment and the second embodiment is that the candidate-waveform selecting processor 140 in a controller 14A is changed to a candidate-waveform selecting processor 140A. The difference between the first embodiment and the second embodiment is that an independent component analyzer 151 and a spectrum analyzer 152 are added to the candidate-waveform selecting processor 140A.

The independent component analyzer 151 selects one-minute heartbeat waveforms indicating near the maximum value of frequency of the average values of the R-R intervals calculated for each one-minute heartbeat waveform according to activity. The independent component analyzer 151 then performs independent component analysis on the one-minute heartbeat waveforms selected according to activity.

For example, the independent component analyzer 151 selects, as the same as the candidate-waveform selecting module 145 does, an appropriate number of one-minute heartbeat waveforms corresponding to average values indicating near the maximum value of frequency using the histogram of average values of R-R intervals according to activity. The independent component analyzer 151 selects one-minute heartbeat waveforms for which the absolute value of the difference in R-R intervals is equal to or smaller than a predetermined threshold t_{TH} as expressed in Expression (1) in the foregoing, as one example. The independent component analyzer 151 performs independent component analysis on the one-minute heartbeat waveforms selected. Such independent component analysis (ICA) is one method of multivariate analysis, and is a calculation method in which signals of information sources are assumed to be independent and signal sources are separated into and extracted as independent components from a signal of a plurality of observed values. In other words, the independent component analyzer 151 uses a plurality of one-minute heartbeat waveforms selected as the observed values and estimates heartbeat waveforms of the signal sources from the one-minute heartbeat waveforms.

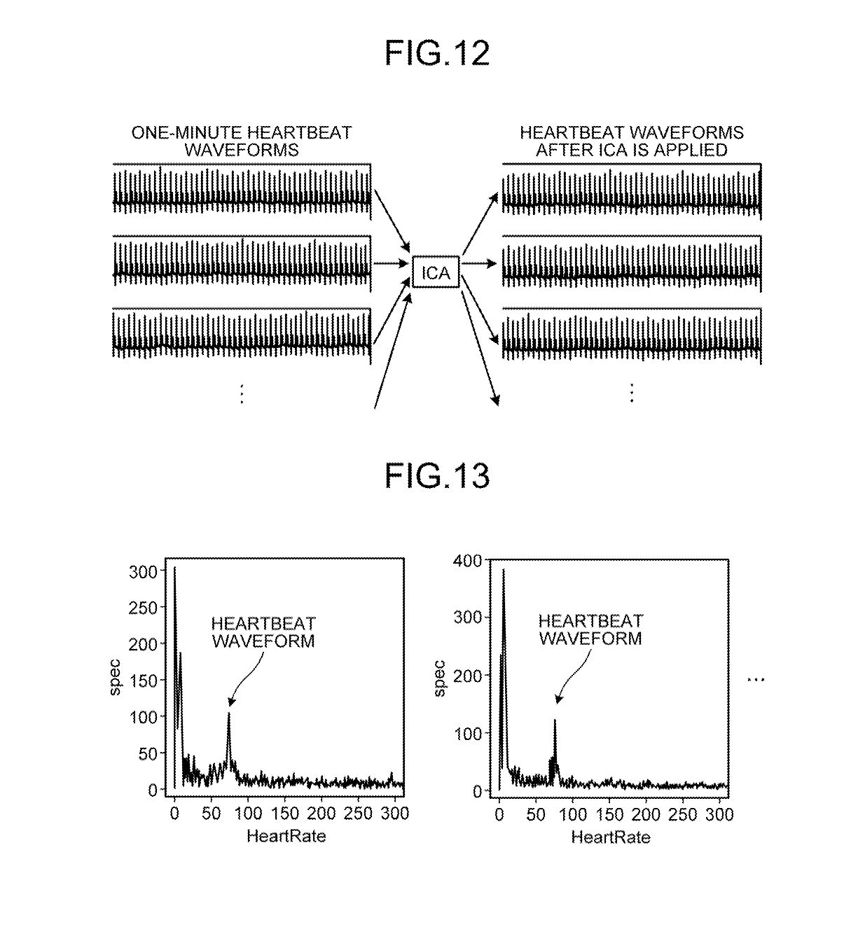

The concept of independent component analysis applied to a plurality of one-minute heartbeat waveforms will now be described with reference to FIG. 12. FIG. 12 is a diagram illustrating the concept of independent component analysis applied to a plurality of one-minute heartbeat waveforms. As illustrated in FIG. 12, the independent component analyzer 151 applies the ICA to a plurality of one-minute heartbeat waveforms selected as the observed values, and generates heartbeat waveforms that are the signal sources.

The spectrum analyzer 152 applies spectral analysis to the heartbeat waveforms to which the independent component analysis has been applied, and selects an appropriate number of heartbeat waveforms in descending order of the peak level of heartbeat for each activity. In other words, the spectrum analyzer 152 generates candidate waveforms for each activity. As for the spectral analysis, fast Fourier transformation is used, for example.

An example of the result of fast Fourier transformation applied to the heartbeat waveforms to which the ICA has been applied will now be described with reference to FIG. 13. FIG. 13 is a diagram illustrating an example of the result of fast Fourier transformation applied to the heartbeat waveforms to which the ICA has been applied. As illustrated in FIG. 13, represented are the heartbeat waveforms that are the result of fast Fourier transformation applied to the heartbeat waveforms to which the ICA has been applied. The peak of the heartbeat waveform is the frequency to represent the heartbeat. The value of the peak increases when the noise included in the heartbeat waveform is low. The spectrum analyzer 152 thus selects an appropriate number of heartbeat waveforms in descending order of the peak value.

### Advantageous Effects of Second Embodiment

In accordance with the second embodiment, the independent component analyzer 151 performs independent component analysis on a plurality of one-minute heartbeat waveforms corresponding to activity. Consequently, the spectrum analyzer 152 can select the candidates of noise-reduced and uniform heartbeat waveforms according to activity. As a result, the representative waveform generator 146 can accurately generate a typical and uniform representative waveform of the focused activity in a unit of one period.

Computer Program and Others

While the biosignal has been described as the electrocardiographic signal in the embodiments, it is not limited to this. The biosignal may be a brain wave signal or a pulse signal, for example, and it only needs to be a cyclic signal concerning a living body. When the biosignal is defined as a brain wave signal, the electrocardiogram sensor 12 only needs to be replaced with a brain wave sensor. When the biosignal is defined as a pulse signal, the electrocardiogram sensor 12 only needs to be replaced with a pulse sensor.

In the embodiments, the activity estimating module 141 estimates the activity of the user from the acceleration detected by the acceleration sensor 11. The calculation of a time constant is then defined to calculate the time constant that defines a stable section from the combination of previous and subsequent activities. However, it is not limited to this, and the activity may be replaced with the intensity of daily activity. In such a case, the activity estimating module 141 only needs to acquire the intensity of daily activity of the user from the acceleration detected by the acceleration sensor 11. The calculation of a time constant only needs to calculate the time constant that defines a stable section from the combination of previous and subsequent intensities of daily activity. For example, the calculation of time constant only needs to detect the transition to a stable section by the standard deviation of the R-R intervals reaching a value equal to or smaller than a threshold from the time when the index of intensity of daily activity is changed from 1.7 (moderate) to 1.3 (low), and define the time elapsed from the time when the index is changed until the detected time as the time constant.

Furthermore, the handheld terminal 1 or 1A can be implemented by installing the functions of various modules such as the acceleration sensor 11, the electrocardiogram sensor 12, the storage module 13, and the controller 14 on a known device such as a mobile computer and a cellular phone.

While the handheld terminal 1 or 1A is defined to include the acceleration sensor 11 and the electrocardiogram sensor 12, either one of the acceleration sensor 11 and the electrocardiogram sensor 12 or the both may be defined as external devices of the handheld terminal 1 or 1A. The sensor of the external device is wirelessly connected to the handheld terminal 1 or 1A, and a given signal is transmitted from a wireless transmitter mounted on the sensor to a receiver mounted on the handheld terminal 1 or 1A.

When either one of the acceleration sensor 11 and the electrocardiogram sensor 12 or the both are defined as the external devices of the handheld terminal 1 or 1A, the handheld terminal 1 or 1A may be defined as a sever. The server only needs to be a known information processing apparatus such as a personal computer and a workstation. Consequently, only the sensors need to be attached to the body of the user, and thus it has advantages in that the activity of the user is not restricted.

Furthermore, when either one of the acceleration sensor 11 and the electrocardiogram sensor 12 or the both are defined as the external devices of the handheld terminal 1 or 1A, the handheld terminal 1 or 1A may be defined as a server in a data center on a cloud. The server only needs to be a known information processing apparatus such as a personal computer and a workstation. This permits the electrocardiographic signals and others of a large number of people to be stored, and thus a database of representative waveforms generated from the stored electrocardiographic signals and others can be made.

The respective constituent elements of the devices illustrated in the drawings are not needed to be physically configured as illustrated in the drawings. In other words, the specific embodiments of distribution or integration of the devices are not limited to those illustrated, and the whole or a part thereof can be configured by being functionally or physically distributed or integrated in any unit according to various types of loads and usage. For example, the stable-section signal extracting module 143 and the R-R interval calculating module 144 may be integrated as a single module. Meanwhile, the candidate-waveform selecting module 145 may be distributed to a first selecting module that selects one-minute heartbeat waveforms indicating near the maximum value of frequency of the average value of R-R intervals and a second selecting module that selects one-minute heartbeat waveforms of low value standard deviations out of the one-minute heartbeat waveforms selected. Furthermore, the storage module 13 such as the electrocardiographic-signal storage module 131 may be connected via a network as an external device of the controller 14.

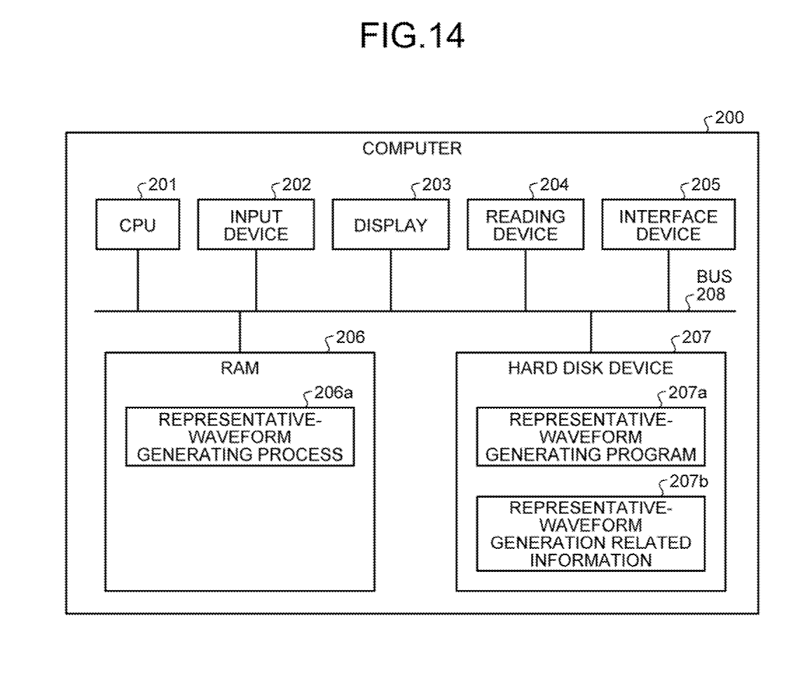

The various processes described in the foregoing embodiments can be implemented by executing a computer program prepared in advance on a computer such as a personal computer and a workstation. In the following description, explained is an example of a computer that executes a representative-waveform generating program that renders the same functions as those of the handheld terminal 1 illustrated in FIG. 1. FIG. 14 is a block diagram illustrating an example of a computer that executes the representative-waveform generating program.

As illustrated in FIG. 14, a computer 200 includes a CPU 201 that executes a variety of arithmetic processes, an input device 202 that receives data input from the user, and a display 203. The computer 200 further includes a reading device 204 that reads out programs and others from a storage medium, and an interface device 205 that exchanges data with other computers via a network. Furthermore, the computer 200 includes a RAM 206 that temporarily stores therein a variety of information and a hard disk device 207. The various devices 201 to 207 are connected to a bus 208.

The hard disk device 207 stores therein a representative-waveform generating program 207a and representative-waveform generation related information 207b. The CPU 201 reads out the representative-waveform generating program 207a and loads it onto the RAM 206. The representative-waveform generating program 207a functions as a representative-waveform generating process 206a.

The representative-waveform generating process 206a corresponds to the activity estimating module 141, the electrocardiographic-signal dividing module 142, the stable-section signal extracting module 143, the R-R interval calculating module 144, the candidate-waveform selecting module 145, and the representative waveform generator 146, for example. The representative-waveform generation related information 207b corresponds to the electrocardiographic-signal storage module 131.

The representative-waveform generating program 207a is not necessarily stored in the hard disk device 207 from the beginning. For example, the program may be stored in a transportable physical medium that is inserted to the computer 200 such as a flexible disk (FD), a CD-ROM, a DVD disc, a magneto-optical disk, and an IC card. The computer 200 may then read out the representative-waveform generating program 207a from the foregoing and execute it.

In accordance with one aspect of the information processing apparatus of the disclosed invention, highly accurate representative waveform data can be generated.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. An information processing apparatus comprising:
a signal dividing module that divides a biosignal into waveforms of a fixed interval;
a calculating module that calculates a plurality of waveform intervals indicative of an interval between adjacent waveforms and calculates an average value of the calculated waveform intervals for each of the waveforms of the fixed interval divided by the signal dividing module; and
a waveform selecting module that selects a plurality of waveforms of the fixed interval corresponding to average values indicating near a maximum value of frequency of average values using the average value of the waveform intervals calculated for each of the waveforms of the fixed interval by the calculating module.

2. The information processing apparatus according to claim 1, further comprising
an activity estimating module that estimates activity of a main body of the biosignal, wherein
the signal dividing module further associates the divided waveforms of the fixed interval with the activity estimated by the activity estimating module, and
the waveform selecting module selects a plurality of waveforms of the fixed interval associated with a single activity.

3. The information processing apparatus according to claim 2, further comprising
a waveform extracting module that extracts waveforms of the fixed interval associated with a subsequent activity out of the waveforms of the fixed interval divided by the signal dividing module after an elapse of a time period indicated by a time constant from a time when a previous activity is changed to the subsequent activity using the time constant indicative of a time period from the time when the previous activity is changed to the subsequent activity until the waveforms stabilize, wherein
the waveform selecting module selects a plurality of waveforms of the fixed interval extracted by the waveform extracting module and associated with the subsequent activity.

4. The information processing apparatus according to claim 3, wherein
the calculating module further calculates standard deviation of waveform intervals for each of the waveforms of the fixed interval from the waveform intervals calculated for each of the waveforms of the fixed interval, and
the waveform selecting module selects a plurality of waveforms of the fixed interval corresponding to average values indicating near the maximum value of frequency of the average values and corresponding to low values of standard deviation out of the standard deviations calculated by the calculating module.

5. The information processing apparatus according to claim 4, further comprising
a time constant calculating module that calculates a time period from a time when a previous activity is changed to a subsequent activity until when standard deviation is equal to or smaller than a threshold as the time constant using the standard deviation of waveform intervals of the waveforms of the fixed interval from the time when the previous activity is changed to the subsequent activity and a result calculated by the calculating module.

6. The information processing apparatus according to claim 3, wherein the waveform selecting module further performs independent component analysis on the selected waveforms of the fixed interval.

7. An information processing apparatus comprising:
a processor; and
a memory, wherein the processor executes:
dividing a biosignal into waveforms of a fixed interval;
calculating a plurality of waveform intervals indicative of an interval between adjacent waveforms and calculating an average value of the calculated waveform intervals for each of the waveforms of the fixed interval divided at the dividing; and
selecting a plurality of waveforms of the fixed interval corresponding to average values indicating near a maximum value of frequency of average values using the average value of the waveform intervals calculated for each of the waveforms of the fixed interval at the calculating.

8. A method for generating representative-waveform generating method on a computer, the method comprising:
dividing a biosignal into waveforms of a fixed interval;
calculating a plurality of waveform intervals indicative of an interval between adjacent R-waves and calculating an average value of the calculated waveform intervals for each of the waveforms of the fixed interval divided at the dividing; and
selecting a plurality of waveforms of the fixed interval corresponding to average values indicating near a maximum value of frequency of average values using the average value of the waveform intervals calculated for each of the waveforms of the fixed interval at the calculating.

9. A non-transitory computer-readable recording medium having stored therein a program for causing a computer to execute a representative-waveform generating process comprising:
dividing a biosignal into waveforms of a fixed interval;
calculating a plurality of waveform intervals indicative of an interval between adjacent R-waves and calculating an average value of the calculated waveform intervals for each of the waveforms of the fixed interval divided at the dividing; and
selecting a plurality of waveforms of the fixed interval corresponding to average values indicating near a maximum value of frequency of average values using the average value of the waveform intervals calculated for each of the waveforms of the fixed interval at the calculating.
